(19) United States
(12) Patent Application Publication
(10) Pub. No.: US 2013/0217935 A1
(43) Pub. Date: Aug. 22, 2013

(54) PROCESS TO MAKE OLEFINS FROM METHANOL AND ISOBUTANOL

(71) Applicants: Cindy Adam, Wierde (BE); Delphine Minoux, Nivelles (BE); Nikolai Nesterenko, Nivelles (BE); Sander Van Donk, Guildford (GB); Jean-Pierre Dath, Beloeil (BE)

(72) Inventors: Cindy Adam, Wierde (BE); Delphine Minoux, Nivelles (BE); Nikolai Nesterenko, Nivelles (BE); Sander Van Donk, Guildford (GB); Jean-Pierre Dath, Beloeil (BE)

(73) Assignee: TOTAL RESEARCH & TECHNOLOGY FELUY, Feluy (BE)

(21) Appl. No.: 13/813,170

(22) PCT Filed: Jul. 8, 2011
(86) PCT No.: PCT/EP2011/061587

§ 371 Date: Apr. 5, 2013

(30) Foreign Application Priority Data
Aug. 3, 2010 (EP) 10171673.6

(51) Int. Cl. C07C 1/22

(52) U.S. Cl. CPC C07C 1/22
USPC 585/314; 585/329; 585/639; 585/640

(57) ABSTRACT

The present invention relates to a process for making essentially ethylene and propylene comprising:
a) providing an alcohol mixture (A) comprising about 20 w % to 100% isobutanol,
b) introducing in a reactor (A) a stream comprising the mixture (A) mixed with methanol or dimethyl ether or mixture thereof, optionally water, optionally an inert component,
c) contacting said stream with a catalyst (A1) in said reactor (A), the MTO reactor, at conditions effective to convert at least a part of the alcohol mixture (A) and at least a part of the methanol and/or dimethyl ether to olefins,
d) recovering from said reactor (A) an effluent comprising: ethylene, propylene, butene, water, optionally unconverted alcohols, various hydrocarbons, and the optional inert component of step b),
e) fractionating said effluent of step d) to produce at least an ethylene stream, a propylene stream, a fraction consisting essentially of hydrocarbons having 4 carbon atoms or more, water and the optional inert component of step a), optionally recycling ethylene in whole or in part at the inlet of the reactor (A), optionally recycling the fraction consisting essentially of hydrocarbons having 4 carbon atoms or more at the inlet of the reactor (A).

### FIELD OF THE INVENTION

The present invention relates to a process to make olefins from methanol and isobutanol.

Olefins are traditionally produced from petroleum feedstocks by catalytic or steam cracking processes. These cracking processes, especially steam cracking, produce light olefin(s), such as ethylene and/or propylene, from a variety of hydrocarbon feedstock. Ethylene and propylene are important commodity petrochemicals useful in a variety of processes for making plastics and other chemical compounds. The limited supply and increasing cost of crude oil has prompted the search for alternative processes for producing hydrocarbon products. The combined MTO/OCP process produces light olefins such as ethylene and propylene as well as heavy hydrocarbons such as butenes which are recycled back or provided for a cracking in OCP. Said MTO process is the conversion of methanol or dimethylether by contact with a molecular sieve. OCP means an olefins cracking process.

The MTO process is a well-known exothermic process to produce light olefins from methanol. Today, the methanol feedstock is mainly produced from fossil resources via syngas route and has to be subjected to purification increasing the manufacturing costs. The heavy oxygenates as well as some hydrocarbons are the main contaminants. Moreover the methanol product containing the heavy oxygenates may be produced on-purpose in a syngas conversion process over fuel alcohols synthesis catalysts. It is advantageous to treat directly such feedstock in MTO reaction without separating the individual compounds. Depending on the respective commercial markets for ethylene and propylene, it may be desirable to vary the ratio of ethylene to propylene formed in MTO and to incorporate into MTO feedstock at least partly renewable product. The extended flexibility in propylene to ethylene ratio can be achieved by modification of heavy alcohol (C2+)/methanol ratio in combination with changes with reaction conditions.

Without wishing to be bound to any theory, the conventional SAPO-34 based MTO is not very effective in conversion of heavy alcohols, especially the oxygenates containing three, four and more carbons. The most noteworthy being ethanol, addition of which increases ethylene production in MTO or in combined MTO/OCP technology. As a result, the propylene to ethylene ratio decreases. However, it is generally recognized that the demand for propylene will increase at a higher pace as compared to ethylene. To address this type of situation, this contribution proposes a co-feeding of the heavy alcohols C230 which contains mainly i-butanol. The non-converted oxygenates or C4+ olefins can be successfully treated over OCP section or recycled back to MTO reactor.

A mixture of the methanol with the heavy oxygenates (C2+) can also be obtained by a simple blending of methanol with heavy oxygenates, comprising isobutanol, preferably derived from biomass. The advantage of that process is to produce at least a part of light olefins from non petroleum sources derived, for example from biomass, in a conventional MTO reactor.

The conversion of heavy oxygenates to hydrocarbons, in particularly the heavy alcohols and ethers (C2+), is a well-known endothermic process. The introduction of higher alcohols in addition to the methanol brings the additional heat integration advantage in respect with highly exothermic MTO reaction.

### BACKGROUND OF THE INVENTION

Isobutanol (2-methyl-1-propanol) has historically found limited applications and its use resembles that of 1-butanol. It has been used as solvent, diluent, wetting agent, cleaner additive and as additive for inks and polymers. Recently, isobutanol has gained interest as fuel or fuel component as it exhibits a high octane number (Blend Octane R+M/2 is 102-103) and a low vapor pressure (RVP is 3.8-5.2 psi).

Isobutanol is often considered as a by-product of the industrial production of 1-butanol (Ullmann's encyclopedia of industrial chemistry, 6^{th} edition, 2002). It is produced from propylene via hydroformylation in the oxo-process (Rh-based catalyst) or via carbonylation in the Reppe-process (Co-based catalyst). Hydroformylation or carbonylation makes n-butanal and iso-butanal in ratios going from 92/8 to 75/25. To obtain isobutanol, the iso-butanal is hydrogenated over a metal catalyst. Isobutanol can also be produced from synthesis gas (mixture of CO, H₂ and CO₂) by a process similar to Fischer-Tropsch, resulting in a mixture of higher alcohols, although often a preferential formation of isobutanol occurs (Applied Catalysis A, general, 186, p. 407, 1999 and Chemiker Zeitung, 106, p. 249, 1982). Still another route to obtain isobutanol, is the base-catalysed Guerbet condensation of methanol with ethanol and/or propanol (J. of Molecular Catalysis A: Chemical 200, 137, 2003 and Applied Biochemistry and Biotechnology, 113-116, p. 913, 2004).

Recently, new biochemical routes have been developed to produce selectively isobutanol from carbohydrates. The new strategy uses the highly active amino acid biosynthetic pathway of microorganisms and diverts its 2-keto acid intermediates for alcohol synthesis. 2-Keto acids are intermediates in amino acid biosynthesis pathways. These metabolites can be converted to aldehydes by 2-keto-acid decarboxylases (KDCs) and then to alcohols by alcohol dehydrogenases (ADHs). Two non-native steps are required to produce alcohols by shunting intermediates from amino acid biosynthesis pathways to alcohol production (Nature, 451, p. 86, 2008 and U.S. patent 2008/0261230). Recombinant microorganisms are required to enhance the flux of carbon towards the synthesis of 2-keto-acids. In the valine biosynthesis 2-ketoisovalerate is on intermediate. Glycolyse of carbohydrates results in pyruvate that is converted into acetolactate by acetolactate synthase. 2,4-dihydroxyisovalerate is formed out of acetolactate, catalysed by isomeroreductase. A dehydratase converts the 2,4-dihydroxyisovalerate into 2-keto-isovalerate. In the next step, a keto acid decarboxylase makes isobutyraldehyde from 2-keto-isovalerate. The last step is the hydrogenation of isobutyraldehyde by a dehydrogenase into isobutanol.

Of the described routes towards isobutanol above, the Guerbet condensation, the synthesis gas hydrogenation and the 2-keto acid pathway from carbohydrates are routes that can use biomass as primary feedstock. Gasification of biomass results in synthesis gas that can be converted into methanol or directly into isobutanol. Ethanol is already at very large scale produced by fermentation of carbohydrates or via direct fermentation of synthesis gas into ethanol. So methanol and ethanol resourced from biomass can be further condensed to isobutanol. The direct 2-keto acid pathway can produce isobutanol from carbohydrates that are isolated from biomass. Simple carbohydrates can be obtained from plants like sugar cane, sugar beet. More complex carbohydrates can be obtained from plants like maize, wheat and other grain bearing plants. Even more complex carbohydrates can be isolated from substantially any biomass, through unlocking of cellulose and hemicellulose from lignocelluloses.

EP 2070896 A1 describes the dehydration of 1-butanol on a porous crystalline aluminosilicate (TON type) in the hydrogen form. At 500° C. the products are in wt %:

| | |
|---|---|
| propylene | 10.76 |
| trans-butene-2 | 16.99 |
| butene-1 | 13.49 |
| isobutene | 31.30 |
| cis-butene-2 | 13.33 |

There is no methanol in the feedstock, only 1-butanol.

U.S. Pat. No. 6,768,037 describes a process for upgrading a Fischer-Tropsch product comprising paraffins, oxygenates (alcohols), and C6+ olefins. The process includes contacting the Fischer-Tropsch product with an acidic olefin cracking catalyst (ZSM-5) to convert the oxygenates and C6+ olefins to form light olefins. The contacting conditions include a temperature in the range of about 500° F. to 850° F., a pressure below 1000 psig, and a liquid hourly space velocity in the range of from about 1 to 20 hr⁻¹. The process further includes recovering the Fischer-Tropsch product comprising unreacted paraffins, and recovering the light olefins. At col 6 lines 16+ is mentioned “. . . The product from a Fischer-Tropsch process contains predominantly paraffins; however, it may also contain C₆₊ olefins, oxygenates, and heteroatom impurities. The most abundant oxygenates in Fischer-Tropsch products are alcohols, and mostly primary linear alcohols. Less abundant types of oxygenates in Fischer-Tropsch products include other alcohol types such as secondary alcohols, acids, esters, aldehydes, and ketones . . . ”. There is no methanol in the feedstock.

WO 2007-149399 relates to a process for making at least one butene comprising contacting a reactant comprising isobutanol and at least about 5% water (by weight relative to the weight of the water plus isobutanol) with at least one acid catalyst at a temperature of about 50° C. to about 450° C. and a pressure from about 0.1 MPa to about 20.7 MPa to produce a reaction product comprising said at least one butene, and recovering said at least one butene from said reaction product to obtain at least one recovered butene. At page 3 is mentionned “. . . The term “butene” includes 1-butene, isobutene, and/or cis and trans 2-butene . . . ”. All the examples are made between 120° C. and 200° C. There is no methanol in the feedstock.

U.S. Pat. No. 4,698,452 relates to a novel process for the conversion of ethanol or its mixtures with light alcohols and optionally water into hydrocarbons with specific and unusual selectivity towards ethylene. More particularly, it relates to the use of ZSM-5 zeolite based catalysts into which Zn alone or Zn and Mn are incorporated. The preferred reaction conditions used in the experiments are as follows: temperature=300° C.-450° C. (most preferred 400° C.); catalyst weight=4 g; total pressure=1 atm; alcohol or aqueous ethanol pressure=0.9 atm; inert gas (stripping gas)=nitrogen; weight hourly space velocity (W.H.S.V.)=2.4 h-1; duration of a run=4 hours. At table 3 dehydration of isobutanol has been made on ZSM-5 (Zn-Mn) and produces paraffins C1-C4, ethylene, propylene, butenes, aromatics and aliphatics. There is no methanol in the feedstock.

JP 2007 290991 A published on 8 Nov. 2007 describes a method for producing olefins in high yield from ethanol as feedstock comprising catalyzing ethanol as the feedstock with a pentasil type-structured zeolite catalyst modified with zirconium and phosphorus. In another version of this method, propylene content of the olefins produced can be controlled by using dimethyl ether and/or methanol together with ethanol as feedstocks and controlling their feed ratio. Examples are as follows:

| | | | |
|---|---|---|---|
| | Comparative | | |
| Example 1 | Example | Example 2 | Example 3 |

Reaction conditions

| | | | | |
|---|---|---|---|---|
| catalyst | 5W—10%Zr/ | HZSM-5 | 5%P—10%Zr/ | 5%P—10%Zr/ |
| | HZSM-5 | | HZSM-5 | HZSM-5 |
| ethanol solution | 4.1 | 4.1 | 4.1 | 3.64 |
| (50 mass %) | | | | |
| dimethyl ether | | | 0.25 | 0.49 |
| temperature ° C. | 500 | 500 | 500 | 500 |
| flow time (hr) | 3 | 1 | 5 | 7 |
| Conversion (%) | 100 | 100 | 100 | 100 |

Yield

| | | | | |
|---|---|---|---|---|
| ethylene | 98.9 | 39.7 | 81.3 | 66.1 |
| propylene (b) | 0.1 | 11.8 | 10.6 | 16.7 |
| butenes | 0.7 | 5.1 | 3.4 | 6.4 |
| aromatic | 0 | 26.1 | 0.3 | 1.4 |
| methane | 0 | 0.1 | tr | tr |
| ethane | tr | 0.3 | tr | tr |
| propane | 0 | 1.2 | tr | 0.1 |
| butane | 0 | 1.3 | 0.1 | 0.3 |
| C₅₊, coke | 0.1 | 14.5 | 4.2 | 8.9 |
| COx | 0 | 0.1 | 0 | 0 |
| diethyl ether | 0 | 0 | 0 | 0 |
| aldehyde | 0.1 | 0 | tr | 0 |
| C₂-C₄ olefins | 99.7 | 56.4 | 95.3 | 89.2 |

Proportions (%)

| | | |
|---|---|---|
| dimethyl ether (a) (%) | 10.8 | 21.2 |
| formation of propylene | 98.1 | 78.8 |
| (b/a) * 100 (%) | | |

U.S. 2006 0161035 A1 describes an MTO reactor coupled with an olefin cracking reactor, also referred to as “heavy olefin interconversion step”. An oxygenate (methanol) is fed to the MTO reactor to produce an effluent stream comprising essentially water, ethylene, propylene and C4+ olefins. Said effluent stream is sent to a fractionation section to recover propylene, ethylene and C430 olefins. Ethylene is recycled to the MTO reactor and the C4+olefins are sent to the olefin cracking reactor. The effluent of the olefin cracking reactor comprising ethylene and propylene is sent to the above fractionation section. The oxygenates cited in this prior art are methanol, dimethyl ether (DME), ethanol, diethyl ether, methylether, formaldehyde, dimethyl ketone, acetic acid, and mixtures thereof. A preferred feedstream contains methanol or dimethylether and mixtures thereof. There is no isobutanol in the feedstock.

U.S. Pat. No. 7,288,689 provides various processes for producing C1 to C4 alcohols, optionally in a mixed alcohol stream, and optionally converting the alcohols to light olefins. In one embodiment, it includes directing a first portion of a syngas stream to a methanol synthesis zone wherein methanol is synthesized. A second portion of the syngas stream is directed to a fuel alcohol synthesis zone wherein fuel alcohol is synthesized. The methanol and at least a portion of the fuel alcohol are directed to an oxygenate to olefin reaction system for conversion thereof to ethylene and propylene. In this prior art “fuel alcohol” means an alcohol-containing composition comprising ethanol, one or more C3 alcohols, one or more C4 alcohols and optionally one or more C5+ alcohols. At col 21 lines 14+ is mentionned “. . . Additionally or alternatively, the fuel alcohol-containing stream comprises one or more C4 alcohols, preferably on the order of from about 0.1 to about 20 weight percent C4 alcohols, preferably from about 1 to about 10 weight percent C4 alcohols, and most preferably from about 2 to about 5 weight percent C4 alcohols, based on the total weight of the fuel alcohol-containing stream. The fuel alcohol-containing stream preferably comprises at least about 5 weight percent C3-C4 alcohols, more preferably at least about 10 weight percent C3-C4 alcohols, and most preferably at least about 15 weight percent C3-C4 alcohols . . . ”. Preferably, the molecular sieve catalyst composition comprises a small pore zeolite or a molecular sieve selected from the group consisting of: MeAPSO, SAPO-5, SAPO-8, SAPO-11, SAPO-16, SAPO-17, SAPO-18, SAPO-20, SAPO-031, SAPO-34, SAPO-35, SAPO-36, SAPO-37, SAPO-40, SAPO-41, SAPO-42, SAPO-44, SAPO-47, SAPO-56, AEI/CHA intergrowths, metal containing forms thereof, intergrown forms thereof, and mixtures thereof.

U.S. Pat. No. 7,199,276 is similar to the previous one but the alcohol other than the methanol is restricted to ethanol.

It has now been discovered that isobutanol or a mixture of isobutanol and other alcohols can be treated together with the methanol feedstock of a MTO/OCP reactors. The propylene production is thereby increased and the energy of the methanol exothermic conversion is used to dehydrate and cracking of isobutanol.

### BRIEF SUMMARY OF THE INVENTION

The present invention relates to a process for making essentially ethylene and propylene comprising:
a) providing an alcohol mixture (A) comprising about 20 w % to 100% isobutanol,
b) introducing in a reactor (A) a stream comprising the mixture (A) mixed with methanol or dimethyl ether or mixture thereof, optionally water, optionally an inert component,
c) contacting said stream with a catalyst (A1) in said reactor (A), the MTO reactor, at conditions effective to convert at least a part of the alcohol mixture (A) and at least a part of the methanol and/or dimethyl ether to olefins,
d) recovering from said reactor (A) an effluent comprising : ethylene, propylene, butene, water, optionally unconverted alcohols, various hydrocarbons, and the optional inert component of step b),
e) fractionating said effluent of step d) to produce at least an ethylene stream, a propylene stream, a fraction consisting essentially of hydrocarbons having 4 carbon atoms or more, water and the optional inert component of step a), optionally recycling ethylene in whole or in part at the inlet of the reactor (A), optionally recycling the fraction consisting essentially of hydrocarbons having 4 carbon atoms or more at the inlet of the reactor (A).

Advantageously, before recycling said hydrocarbons having 4 carbon atoms or more at the inlet of the reactor (A), said hydrocarbons having 4 carbon atoms or more are sent to a second fractionator to purge the heavies.

In an embodiment the alcohol feed is subjected to purification to reduce the content in the metal ions, in more particularly in Na, Fe, K, Ca and Al.

In a specific embodiment the alcohol mixture (A) comprises 40 to 100w % of isobutanol.

In a specific embodiment the alcohol mixture (A) comprises 60 to 100 w % of isobutanol.

In a specific embodiment the alcohol mixture (A) comprises 80 to 100 w % of isobutanol.

In a specific embodiment the alcohol mixture (A) comprises essentially isobutanol.

Advantageously the alcohol mixture (A) comprises, in addition of isobutanol, C2+ alcohols.

Advantageously the feedstock of the MTO reactor, comprising the alcohol mixture (A) and the methanol or dimethylether, comprises at least 50 w % of MeOH and/or dimethylether.

It would not depart from the scope of the invention to replace alcohols in whole or in part by the corresponding ethers and/or corresponding aldehydes.

In an embodiment the process further comprises:
f) introducing at least a part of the fraction consisting essentially of hydrocarbons having 4 carbon atoms or more in an OCP reactor (also called Olefin Cracking Process),
g) contacting said stream in said OCP reactor with a catalyst which is selective towards light olefins in the effluent, to produce an effluent with an olefin content of lower molecular weight than that of the feedstock,
h) fractionating said effluent of step g) to produce at least an ethylene stream, a propylene stream and a fraction consisting essentially of hydrocarbons having 4 carbon atoms or more,
optionally recycling ethylene in whole or in part at the inlet of the OCP reactor of step g), or at the inlet of the reactor (A) or at the inlet of both the OCP reactor of step f) and the reactor (A),
optionally recycling the fraction consisting essentially of hydrocarbons having 4 carbon atoms or more at the inlet of the OCP reactor.

In an embodiment a part of the alcohol mixture (A) comprising about 20 w % to 100% isobutanol is injected to OCP reactor without being treated over the MTO section.

Optionally water is removed from the feedstock sent to the OCP reactor.

In an embodiment the catalyst in the MTO reactor contains at least one cracking component comprising molecular sieve containing advantageously at least one 10 members ring into the structure and selected from the group of crystalline silicoaluminates (zeolite), metal-exchanged zeolite, phosphated zeolite, silicoalumophosphate or a mixture of thereof. The presence of the cracking component leads to cracking the heavy hydrocarbons originated from primary dehydration reaction in MTO conversion zone (the absence of such cracking components limit the conversion of heavy oxygenates to light olefins in a conventional small-pore silicoalumophosphate based MTO). On the contrary, the small-pore molecular sieve can be present also in the MTO conversion zone together with the cracking component. These latter combined silicon, aluminium, and phosphorous based molecular sieves have been described in detail in publication, including, for example, WO2009/016154.

Without wishing to be bound to any theory, the cracking component can be defined as a catalyst which is able to convert C4+ heavy olefinic hydrocarbon to ethylene and propylene. On the contrary, the conventional MTO catalyst based on small-pore SAPO molecular sieve has only limited capability in such transformation. Therefore, the presence of the cracking component in the MTO conversion zone has a marked effect on the activity in transformation of heavy-alcohol containing feedstock rich in isobutanol. Typically, this cracking component is also active and useful in methanol conversion and can be used as a MTO catalyst itself. However, for a selectivity reason, said cracking component may be used as a co-catalyst in combination with a conventional small-pore silicoaluminophosphate molecular sieve in the MTO conversion zone.

The other advantages of the catalyst are low deactivation rate and lower coke selectivity from heavy oxygenates due to better diffusion restriction. This property allows maximizing the bio-carbon content in the monomers extracted from MTO effluent.

Optionally, the feedstock containing oxygenates (methanol and isobutanol stream) can be subjected to a conversion in MTO reactor in presence of steam. That means that the feedstock containing the methanol and heavy oxygenates may contain some water before injection to the MTO conversion zone. The heavy oxygenates derived from bio-mass as well as the feedstock produced from syngas may contain a lot of water. The complete drying of these compounds is extremely energy demanding process. The idea is to feed directly the water-congaing bio-feedstock to MTO zone without complete water extraction. In preferred embodiment some water could be extracted on-spot from bio-oxygenates to reduce the transportation/logistic costs.

The selectivity to ethylene and propylene in an “Oxygenates to Olefins” reaction zone may vary depending on reaction conditions but the flexibility of propylene to ethylene ratio is limited. By varying the ratio of methanol and heavy oxygenates, the selectivity to propylene & ethylene may be tuned without changing the reaction conditions.

Advantageously, the alcohols of the mixture (A) are derived from the biomass and thus it gives the opportunities to introduce a part of renewable carbon in the light olefin product.

Advantageously isobutanol, as well as other alcohols, are obtained by fermentation of carbohydrates coming from the biomass, or from the syngas route or from the base-catalysed Guerbet condensation.

In an embodiment isobutanol is produced by the direct 2-keto acid pathway from carbohydrates that are isolated from biomass.

One skilled in the art will also appreciate that the olefin products made by the present invention can be polymerized, optionally with comonomers, to form polyolefins, particularly polyethylenes and polypropylenes.

### DETAILED DESCRIPTION OF THE INVENTION

As regards the stream introduced at step b) the inert component is any component provided there is no adverse effect on the catalyst. By way of examples the inert component is selected among the saturated hydrocarbons having up to 10 carbon atoms, naphtenes, nitrogen and CO2. An example of inert component can be any individual saturated compound, a synthetic mixture of the individual saturated compounds as well as some equilibrated refinery streams like straight naphtha, butanes etc. Advantageously it is a saturated hydrocarbon or a mixture of saturated hydrocarbons having from 3 to 7 carbon atoms, more advantageously having from 4 to 6 carbon atoms and is preferably pentane. The weight proportions of respectively alcohols, water and inert component are, for example, 5-100/0-95/0-95 (the total being 100). The stream (A) can be liquid or gaseous.

As regards the reactor (A), it can be a fixed bed reactor, a moving bed reactor or a fluidized bed reactor. A typical fluid bed reactor is one of the FCC type used for fluidized-bed catalytic cracking in the oil refinery. A typical moving bed reactor is of the continuous catalytic reforming type. The reaction may be performed continuously in a fixed bed reactor configuration using a pair of parallel “swing” reactors. The various preferred catalysts of the present invention have been found to exhibit high stability. This enables the MTO process to be performed continuously in two parallel “swing” reactors wherein when one reactor is operating, the other reactor is undergoing catalyst regeneration. The catalyst in the present invention also can be regenerated several times.

As regards the catalyst (A1) of step c), it can be any catalyst capable to convert at least a part of the alcohol mixture (A) and at least a part of the methanol and/or dimethyl ether to olefins. One can cite, zeolites, modified zeolites (including Me-modified & P-modified zeolites) and silicoalumophosphates or a mixture thereof.

Advantageously the catalyst in the MTO reactor contains at least one cracking component comprising molecular sieve containing advantageously at least one 10 members ring into the structure and selected from a group of crystalline silicoaluminates (zeolite), metal-exchanged zeolite, phosphated zeolite, silicoalumophosphate or a mixture of thereof. The presence of a cracking component leads to cracking the heavy hydrocarbons originated from primary dehydration reaction in MTO conversion zone (the absence of such components limit the conversion of heavy oxygenates to light olefins in a conventional small-pore silicoalumophosphate based MTO). On the contrary, the small-pore molecular sieve can be present also in the MTO conversion zone together with the cracking component. These latter combined silicon, aluminum, and phosphorous based molecular sieves have been described in detail in publication, including, for example, WO2009/016154.

According to an embodiment the cracking component of the catalyst (A1) is a crystalline Porous Aluminophosphate containing advantageously at least one 10 and/or 12 members ring into the structure.

The porous crystalline aluminophosphate may be one that is comprised of aluminum and phosphorus that are partly substituted by silicon, boron, Ni, Zn, Mg, Mn such as a porous crystalline metalaluminophosphate. The structure of such crystalline porous aluminophosphates may, for example, be those that are identified by codes for zeolites described above as AEL, AFI, AFO or FAU.

The above porous crystalline aluminophosphate is preferably a porous crystalline silicoaluminophosphate. Specifically, SAPO5, and the like having an AFI structure, SAPO41, and the like having an AFO structure, SAPO11, and the like having an AEL structure, structure or SAPO37, and the like having a FAU structure may be mentioned.

According to another specific embodiment, suitable catalysts for the present process is the silicoaluminophosphate molecular sieves, in particular of the AEL group with typical example the SAPO-11 molecular sieve. The SAPO-11 molecular sieve is based on the ALPO-11, having essentially an Al/P ratio of 1 atom/atom. During the synthesis silicon precursor is added and insertion of silicon in the ALPO framework results in an acid site at the surface of the micropores of the 10-membered ring sieve. The silicon content ranges from 0.1 to 10 atom % (Al+P+Si is 100).

Various commercial zeolite products nay be used, or it is possible to use zeolites that have been synthesized by a known method disclosed in e.g.

“Verified Synthesis of Zeolitic Materials” (2^{nd} Revised Edition 2001 Elsevier) published by the above IZA.

According to an embodiment the cracking component of catalyst (Al) is a crystalline silicate containing advantageously at least one 10 members ring into the structure. It is by way of example of the MFI (ZSM-5, silicalite-1, boralite C, TS-1), MEL (ZSM-11, silicalite-2, boralite D, TS-2, SSZ-46), FER (Ferrierite, FU-9, ZSM-35), MTT (ZSM-23), WNW (MCM-22, PSH-3, ITQ-1, MCM-49), EUO (ZSM-50, EU-1), MFS (ZSM-57), CON (CIT-1) and ZSM-48 family of microporous materials consisting of silicon, aluminium, oxygen and optionally boron. Advantageously in said first embodiment the catalyst (A1) is a crystalline silicate, metal containing crystalline silicate or a dealuminated crystalline silicate.

The crystalline silicate can have a ratio Si/Al of at least about 100 and is advantageously selected among the MFI and the MEL and modified with the metals Mg, Ca, La, Ni, Ce, Zn, Co, Ag, Fe, Cu. The metal content is at least 0.1wt %.

The dealuminated crystalline silicate is advantageously such as about 10% by weight of the aluminium is removed. Such dealumination is advantageously made by a steaming optionally followed by a leaching.

In another specific embodiment the crystalline silicate catalyst is mixed with a binder, preferably an inorganic binder, and shaped to a desired shape, e.g. pellets. The binder is selected so as to be resistant to the temperature and other conditions employed in the dehydration process of the invention. The binder is an inorganic material selected from clays, silica, metal silicate, metal borates, metal oxides such as ZrO₂ and/or metals, or gels including mixtures of silica and metal oxides.

In an embodiment it can be a crystalline alumosilicate of the MFI family or the MEL family. An example of MFI silicates is ZSM-5. An example of an MEL zeolite is ZSM-11 which is known in the art. Other examples are described by the International Zeolite Association (Atlas of Zeolite Structure Types, 1987, Butterworths).

Crystalline silicates are microporous crystalline inorganic polymers based on a framework of XO₄ tetrahydra linked to each other by sharing of oxygen ions, where X may be trivalent (e.g. Al, B, . . . ) or tetravalent (e.g. Ge, Si, . . . ). The crystal structure of a crystalline silicate is defined by the specific order in which a network of tetrahedral units are linked together. The size of the crystalline silicate pore openings is determined by the number of tetrahedral units, or, alternatively, oxygen atoms, required to form the pores and the nature of the cations that are present in the pores. They possess a unique combination of the following properties: high internal surface area; uniform pores with one or more discrete sizes; ion exchangeability; good thermal stability; and ability to adsorb organic compounds. Since the pores of these crystalline alumosilicates are similar in size to many organic molecules of practical interest, they control the ingress and egress of reactants and products, resulting in particular selectivity in catalytic reactions. Crystalline alumosilicates with the MFI structure possess a bi-directional intersecting pore system with the following pore diameters: a straight channel along [010]: 0.53-0.56 nm and a sinusoidal channel along [100]: 0.51-0.55 nm. Crystalline alumosilicates with the MEL structure possess a bi-directional intersecting straight pore system with straight channels along [100] having pore diameters of 0.53-0.54 nm.

According to an embodiment the the cracking component of catalyst (A1) is a P-modified zeolite (Phosphorus-modified zeolite). Said phosphorus modified molecular sieves can be prepared based on MFI, MOR, MEL, clinoptilolite or FER MWW, TON, EUO, MFS and ZSM-48 family of microporous molecular sieves having an initial Si/Al ratio advantageously between 4 and 500. The P-modified zeolites of this recipe can be obtained based on cheap crystalline silicates with low Si/Al ratio (below 30).

By way of example said P-modified zeolite is made by a process comprising in that order:
selecting a zeolite (advantageously with Si/Al ratio between 4 and 500) among H⁺ or NH₄⁺-form of MFI, MEL, FER, MOR, clinoptilolite, MWW, EUO, MFS and ZSM-48;
introducing P at conditions effective to introduce advantageously at least 0.05 wt % of P;
separation of the solid from the liquid if any;
an optional washing step or an optional drying step or an optional drying step followed by a washing step;
a calcination step.

The zeolite with low Si/Al ratio has been made previously with or without direct addition of an organic template.

Optionally the process to make said P-modified zeolite comprises the steps of steaming and leaching. The method consists in steaming followed by leaching. It is generally known by the persons in the art that steam treatment of zeolites, results in aluminium that leaves the zeolite framework and resides as aluminiumoxides in and outside the pores of the zeolite. This transformation is known as dealumination of zeolites and this term will be used throughout the text. The treatment of the steamed zeolite with an acid solution results in dissolution of the extra-framework aluminiumoxides. This transformation is known as leaching and this term will be used throughout the text. Then the zeolite is separated, advantageously by filtration, and optionally washed. A drying step can be envisaged between filtering and washing steps. The solution after the washing can be either separated, by way of example, by filtering from the solid or evaporated.

P can be introduced by any means or, by way of example, according to the recipe described in U.S. Pat. No. 3,911,041, U.S. Pat. No. 5,573,990 and U.S. Pat. No. 6,797,851.

The catalyst made of a P-modified zeolite can be the P-modified zeolite itself or it can be the P-modified zeolite formulated into a catalyst by combining with other materials that provide additional hardness or catalytic activity to the finished catalyst product. Advantageously, at least a part of phosphorous is introduced into zeolite before shaping. In a specific embodiment, the formed P-precursor can be further modified with the metals selected from Mg, Ca, La, Ni, Ce, Zn, Co, Ag, Fe, Cu according to the recipe described in WO 09092779 and WO 09092781.

The separation of the liquid from the solid is advantageously made by filtering at a temperature between 0-90° C., centrifugation at a temperature between 0-90° C., evaporation or equivalent.

Optionally, the zeolite can be dried after separation before washing. Advantageously said drying is made at a temperature between 40-600° C., advantageously for 1-10 h. This drying can be processed either in a static condition or in a gas flow. Air, nitrogen or any inert gases can be used.

The washing step can be performed either during the filtering (separation step) with a portion of cold (<40° C.) or hot water (>40 but <90° C.) or the solid can be subjected to a water solution (1 kg of solid/4 liters water solution) and treated under reflux conditions for 0.5-10 h followed by evaporation or filtering.

Final equilibration step is performed advantageously at the temperature 400-800° C. in presence of steam for 0.01-48h. Advantageously the steam partial pressure is at least 0.1 bars. Air, nitrogen or any inert gases can be fed together with steam.

According to a specific embodiment the phosphorous modified zeolite is made by a process comprising in that order:
selecting a zeolite (advantageously with Si/AI ratio between 4 and 500, from 4 to 30 in a specific embodiment) among H⁺ or NH₄⁺-form of MFI, MEL, FER, MOR, clinoptilolite, MWW, TON, EUO, MFS and ZSM-48;
steaming at a temperature ranging from 400 to 870° C. for 0.01-200 h;
leaching with an aqueous acid solution at conditions effective to remove a substantial part of Al from the zeolite;
introducing P with an aqueous solution containing the source of P at conditions effective to introduce advantageously at least 0.05 wt % of P;
separation of the solid from the liquid;
an optional washing step or an optional drying step or an optional drying step followed by a washing step;
a calcination step.

Optionally between the steaming step and the leaching step there is an intermediate step such as, by way of example, contact with silica powder and drying.

Optionally the leaching and introducing P are made simultaneously by using an acid mixture comprising phosphorus to make the leaching.

Advantageously the selected MFI, MEL, FER, MOR, clinoptilolite, MWW, EUO, MFS and ZSM-48 (or H⁺ or NH₄⁺-form MFI, MEL, FER, MOR, clinoptilolite, MWW, EUO, MFS and ZSM-48) has an initial atomic ratio Si/Al of 100 or lower and from 4 to 30 in a specific embodiment. The conversion to the H⁺ or NH₄⁺-form is known per se and is described in U.S. Pat. No. 3,911,041 and U.S. Pat. No. 5,573,990.

Advantageously the final P-content is at least 0.05 wt % and preferably between 0.3 and 7 w %. Advantageously at least 10% of Al, in respect to parent zeolite MFI, MEL, FER, MOR and clinoptilolite, MWW, EUO, MFS and ZSM-48, have been extracted and removed from the zeolite by the leaching.

Then the zeolite either is separated from the washing solution or is dried without separation from the washing solution. Said separation is advantageously made by filtration. Then the zeolite is calcined, by way of example, at 400° C. for 2-10 hours.

In the steam treatment step, the temperature is preferably from 420 to 870° C., more preferably from 480 to 760° C. The pressure is preferably atmospheric pressure and the water partial pressure may range from 13 to 100 kPa. The steam atmosphere preferably contains from 5 to 100 vol % steam with from 0 to 95 vol % of an inert gas, preferably nitrogen. The steam treatment is preferably carried out for a period of from 0.01 to 200 hours, advantageously from 0.05 to 200 hours, more preferably from 0.05 to 50 hours. The steam treatment tends to reduce the amount of tetrahedral aluminium in the crystalline silicate framework by forming alumina.

The leaching can be made with an organic acid such as citric acid, formic acid, oxalic acid, tartaric acid, malonic acid, succinic acid, glutaric acid, adipic acid, maleic acid, phthalic acid, isophthalic acid, fumaric acid, nitrilotriacetic acid, hydroxyethylenediaminetriacetic acid, ethylenediaminetetracetic acid, trichloroacetic acid trifluoroacetic acid or a salt of such an acid (e.g. the sodium salt) or a mixture of two or more of such acids or salts. The other inorganic acids may comprise an inorganic acid such as nitric acid, hydrochloric acid, methansulfuric acid, phosphoric acid, phosphonic acid, sulfuric acid or a salt of such an acid (e.g. the sodium or ammonium salts) or a mixture of two or more of such acids or salts.

The residual P-content is adjusted by P-concentration in the aqueous acid solution containing the source of P, drying conditions and a washing procedure if any. A drying step can be envisaged between filtering and washing steps.

Said P-modified zeolite can be used as itself as a catalyst. In another embodiment it can be formulated into a catalyst by combining with other materials that provide additional hardness or catalytic activity to the finished catalyst product. Materials which can be blended with the P-modified zeolite can be various inert or catalytically active materials, or various binder materials. These materials include compositions such as kaolin and other clays, various forms of rare earth metals, phosphates, alumina or alumina sol, titanic, zirconia, quartz, silica or silica sol, and mixtures thereof. These components are effective in densifying the catalyst and increasing the strength of the formulated catalyst. The catalyst may be formulated into pellets, spheres, extruded into other shapes, or formed into a spray-dried particles. The amount of P-modified zeolite which is contained in the final catalyst product ranges from 10 to 90 weight percent of the total catalyst, preferably 20 to 70 weight percent of the total catalyst.

Final equilibration step is performed advantageously at the temperature 400-800° C. in presence of steam for 0.01-48h . Advantageously the steam partial pressure is at least 0.1 bars. Air, nitrogen or any inert gases can be fed together with steam.

A catalyst has already been described in WO2009016153, WO2009092779, WO2009092781.

The other catalyst components of the catalyst (A1) could be binders, fillers or other catalytically active materials. These materials are typically effective in reducing overall catalyst cost, acting as thermal sinks assisting in shielding heat from the catalyst composition for example during regeneration, densifying the catalyst composition, increasing catalyst strength such as crush strength and attrition resistance, and controlling the rate of conversion.

Clays, modified clays, basic compounds, transition metal-containing compounds as well as small pore-zeolites and silicoaluminophosphates may be implemented as other catalytically active materials. Advantageously, the small pore molecular sieves can be selected from the group of CHA, AEI, LEV, ERI or a mixture of thereof including intergrowth phases.

As regards the operating conditions of the MTO reactor, the pressure is advantageously 5 barg (bar gauge) or less and preferably between 0 and 5 barg more preferably around the atmospheric pressure. The temperature can be between 400° C. and 600° C., and the WHSV between 0.01 and 100 h-1.

As regards step e), the fractionation of said effluent of step c) said fractionation is carried out by any means, they are known per se.

As regards the reaction in step g), it is referred as an “OCP process”. It can be any catalyst provided it is selective to light olefins. Said OCP process is known per se. It has been described in EP 1036133, EP 1035915, EP 1036134, EP 1036135, EP 1036136, EP 1036138, EP 1036137, EP 1036139, EP 1194502, EP 1190015, EP 1194500 and EP 1363983 the content of which are incorporated in the present invention.

The catalysts used in the MTO reactor and the OCP reactor can be the same or different.

The catalyst can be selected among the catalysts (A1) of step c) above and is employed under particular reaction conditions whereby the catalytic cracking of the C₄⁺ olefins readily proceeds. Different reaction pathways can occur on the catalyst. Olefinic catalytic cracking may be understood to comprise a process yielding shorter molecules via bond breakage.

Should the water is not removed or should a substantial amount of water remains in the feed of step g) it is recommended to use in said OCP reactor a catalyst able to operate in the presence of water. Advantageously said catalyst is a P-modified zeolite as explained above in the description of the catalyst (A1).

In the catalytic cracking process of the OCP reactor, the process conditions are selected in order to provide high selectivity towards propylene or ethylene, as desired, a stable olefin conversion over time, and a stable olefinic product distribution in the effluent. Such objectives are favoured with a low pressure, a high inlet temperature and a short contact time, all of which process parameters are interrelated and provide an overall cumulative effect.

The process conditions are selected to disfavour hydrogen transfer reactions leading to the formation of paraffin's, aromatics and coke precursors. The process operating conditions thus employ a high space velocity, a low pressure and a high reaction temperature. The LHSV ranges from 0.5 to 30 hr⁻¹, preferably from 1 to 30 hr⁻¹. The olefin partial pressure ranges from 0.1 to 2 bars, preferably from 0.5 to 1.5 bars (absolute pressures referred to herein). A particularly preferred olefin partial pressure is atmospheric pressure (i.e. 1 bar). The feedstock is preferably fed at a total inlet pressure sufficient to convey the feedstocks through the reactor. Said feedstock may be fed undiluted or diluted in an inert gas, e.g. nitrogen or steam. Preferably, the total absolute pressure in the reactor ranges from 0.5 to 10 bars. The use of a low olefin partial pressure, for example atmospheric pressure, tends to lower the incidence of hydrogen transfer reactions in the cracking process, which in turn reduces the potential for coke formation which tends to reduce catalyst stability. The cracking of the olefins is preferably performed at an inlet temperature of the feedstock of from 400° to 650° C., more preferably from 450° to 600° C., yet more preferably from 540° C. to 590° C. In order to maximize the amount of ethylene and propylene and to minimize the production of methane, aromatics and coke, it is desired to minimize the presence of diolefins in the feed. Diolefin conversion to monoolefin hydrocarbons may be accomplished with a conventional selective hydrogenation process such as disclosed in U.S. Pat. No. 4,695,560 hereby incorporated by reference.

The OCP reactor can be a fixed bed reactor, a moving bed reactor or a fluidized bed reactor. A typical fluid bed reactor is one of the FCC type used for fluidized-bed catalytic cracking in the oil refinery. A typical moving bed reactor is of the continuous catalytic reforming type. As described above, the process may be performed continuously using a pair of parallel “swing” reactors. The cracking process is endothermic; therefore, the reactor should be adapted to supply heat as necessary to maintain a suitable reaction temperature. Several reactors may be used in series with interheating between the reactors in order to supply the required heat to the reaction. Each reactor does a part of the conversion of the feedstock. Online or periodic regeneration of the catalyst may be provided by any suitable means known in the art.

The various preferred catalysts of the OCP reactor have been found to exhibit high stability, in particular being capable of giving a stable propylene yield over several days, e.g. up to ten days. This enables the olefin cracking process to be performed continuously in two parallel “swing” reactors wherein when one reactor is in operation, the other reactor is undergoing catalyst regeneration. The catalyst can be regenerated several times.

As regards step h) and the effluent of OCP reactor of step g), said effluent comprises methane, ethylene, propylene, optionally the inert component and hydrocarbons having 4 carbon atoms or more. Advantageously said OCP reactor effluent is sent to a fractionator and the light olefins (ethylene and propylene) are recovered. Advantageously the hydrocarbons having 4 carbon atoms or more are recycled at the inlet of the OCP reactor. Advantageously, before recycling said hydrocarbons having 4 carbon atoms or more at the inlet of the OCP reactor, said hydrocarbons having 4 carbon atoms or more are sent to a second fractionator to purge the heavies.

Optionally, in order to adjust the propylene to ethylene ratio, ethylene in whole or in part can be recycled over the OCP reactor and advantageously converted into more propylene. Ethylene can also be recycled in whole or in part at the inlet of the reactor (A).

### EXAMPLES

### Example 1 (according to the invention)

The catalyst is a phosphorous modified zeolite (P-ZSM5), prepared according to the following recipe. A sample of zeolite ZSM-5 (Si/Al=13) in H-form was steamed at 550° C. for 6 h in 100% H₂O. Then, 600 g of the steamed solid was subjected to a contact with 114 g of an aqueous solution of H₃PO₄ (85% wt) for 2 h under reflux condition (4 ml/1 g zeolite) followed by addition of 35 g of CaCO3. Then the solution was dried by evaporation under rigours stirring for 3 days at 80° C. 720 g of the dried sample was extruded with 356 g of Bindzil and 3 wt % of extrusion additives. The extruded solid was dried at 110° C. for 16 h and steamed at 600° C. for 2 h.

Catalyst tests were performed on 0.8 g of catalyst grains (35-45 meshes) loaded in the tubular fixed bed reactor. The feedstock which contains isobutanol/methanol mixture having the 15/85 wt % composition has been processed on the catalyst under 1.3 bara, at temperatures 550° C., and with WHSV-4 and 10 h⁻¹ based on total feed. The results are in tables 1 hereunder. The values in the tables are given in the weight percent on carbon basis, coke free basis and represent an average catalyst performance during 20 h TOS. The data given below illustrate a performance of P-ZSM-5 disclosing in this invention in conversion of mixed Methanol/i-Butanol feedstock to propylene and ethylene in combined MTO/OCP process.

TABLE 1

| Feed | MeOH + iBuOH | |
|---|---|---|
| Tᵢₙ, ° C. | 550 | 550 |
| P, bara | 1.2 | 1.2 |
| WHSV (MeOH), h⁻¹ | 3.4 | 8.5 |
| WHSV, i-BuOH, h⁻¹ | 0.60 | 1.50 |
| Conversion, % | 99.5 | 99.5 |

Yield on CH2 basis, %

| | | |
|---|---|---|
| C1 (Methane) | 0.6 | 0.6 |
| Paraffins (n + i + CyP) | 8.8 | 7.1 |
| Olefins (n + i + CyO) | 86.0 | 89.3 |
| Aromatics (A) | 4.7 | 3.1 |
| Ethane | 0.2 | 0.1 |
| Propane | 1.6 | 0.7 |
| Purity C2's | 98.2 | 98.8 |
| Purity C3's | 95.8 | 98.1 |
| C3═/C2═ | 3.5 | 5.4 |
| C2═ + C3═ | 48.3 | 46.8 |
| Ethylene | 11.0 | 7.6 |
| Propylene | 37.3 | 39.1 |
| OCP feed (non cyclic olefins | 36.4 | 40.5 |
| C4+) | | |

MTO + OCP (simulated values)

| | | |
|---|---|---|
| Ethylene | 16.3 | 13.5 |
| Propylene | 62.3 | 66.9 |
| C3═/C2═ | 3.8 | 4.9 |
| C2═ + C3═ | 78.5 | 80.4 |

1. Process for making essentially ethylene and propylene comprising:
a) providing an alcohol mixture (A) comprising about 20 w % to 100% isobutanol,
b) introducing in a reactor (A) a stream comprising the mixture (A) mixed with methanol or dimethyl ether or mixture thereof, optionally water, optionally an inert component,
c) contacting said stream with a catalyst (Al) in said reactor (A), the MTO reactor, at conditions effective to convert at least a part of the alcohol mixture (A) and at least a part of the methanol and/or dimethyl ether to olefins,
d) recovering from said reactor (A) an effluent comprising: ethylene, propylene, butene, water, optionally unconverted alcohols, various hydrocarbons, and the optional inert component of step b),
e) fractionating said effluent of step d) to produce at least an ethylene stream, a propylene stream, a fraction consisting essentially of hydrocarbons having 4 carbon atoms or more, water and the optional inert component of step a),
optionally recycling ethylene in whole or in part at the inlet of the reactor (A),
optionally recycling the fraction consisting essentially of hydrocarbons having 4 carbon atoms or more at the inlet of the reactor (A).

2. Process according to claim 1 wherein the alcohol feed is subjected to purification to reduce the content in the metal ions, in more particularly in Na, Fe, K , Ca and Al.

3. Process according to claim 1 wherein the catalyst (Al) is selected among the crystalline silicates.

4. Process according to claim 1 wherein the catalyst (A1) is a P-modified zeolite.

5. Process according to claim 1 wherein the temperature in the MTO reactor ranges from 400° C. to 600° C.

6. Process according to claim 1 wherein the pressure of the MTO reactor is 5 barg (bar gauge) or less.

7. Process according to claim 1 wherein the pressure of the MTO reactor is around the atmospheric pressure.

8. Process according to claim 1 wherein the alcohol mixture (A) comprises 40 to 100w % of isobutanol.

9. Process according to claim 1 wherein the alcohol mixture (A) comprises 60 to 100w % of isobutanol.

10. Process according to claim 1 wherein the alcohol mixture (A) comprises 80 to 100w % of isobutanol.

11. Process according to claim 1 wherein the alcohol mixture (A) is essentially isobutanol.

12. Process according to claim 1 further comprising:
f) introducing at least a part of the fraction consisting essentially of hydrocarbons having 4 carbon atoms or more in an OCP reactor (also called Olefin Cracking Process),
g) contacting said stream in said OCP reactor with a catalyst which is selective towards light olefins in the effluent, to produce an effluent with an olefin content of lower molecular weight than that of the feedstock,
h) fractionating said effluent of step g) to produce at least an ethylene stream, a propylene stream and a fraction consisting essentially of hydrocarbons having 4 carbon atoms or more, optionally recycling ethylene in whole or in part at the inlet of the OCP reactor of step g), or at the inlet of the reactor (A) or at the inlet of both the OCP reactor of step f) and the reactor (A), optionally recycling the fraction consisting essentially of hydrocarbons having 4 carbon atoms or more at the inlet of the OCP reactor

13. Process according to claim 1 wherein the cracking of the olefins in the OCP reactor is performed at an inlet temperature of the feedstock temperature of the OCP reactor from 400° C. to 650° C.

14. Process according to claim 1 wherein the cracking of the olefins in the OCP reactor is performed at an inlet temperature of the feedstock temperature of the OCP reactor from 450° C. to 600° C.

15. Process according to claim 1 wherein the cracking of the olefins in the OCP reactor is performed at an inlet temperature of the feedstock temperature of the OCP reactor from 540° C. to 590° C.

16. Process according to claim 1 wherein water is removed from the feedstock sent to the OCP reactor.

17. Process according to claim 1 wherein isobutanol is obtained by fermentation of carbohydrates coming from the biomass, or from the syngas route or from the base-catalysed Guerbet condensation.

18. Process according to claim 1 wherein isobutanol is produced by the direct 2-keto acid pathway from carbohydrates that are isolated from biomass.

19. Process according to claim 1 wherein ethylene is further polymerized optionally with one or more comonomers.
